(19) United States
(12) Patent Application Publication
(10) Pub. No.: US 2003/0030015 A1
(43) Pub. Date: Feb. 13, 2003

(54) Apparatus for irradiating articles

(76) Inventor: Alex Waluszko, Upland, CA (US)

Correspondence Address: James E. Brunton, 700 North Brand Blvd., Suite 860, P.O. Box 29000, Glendale, CA, 91029, US

(21) Appl. No.: 09/927,251

(22) Filed: Aug. 10, 2001

(51) Int. Cl. A61N 5/00

(52) U.S. Cl. 250/503.1

(57) ABSTRACT

A method and apparatus for precisely controlling the area and accuracy of total radiation dose exposure of an article to be irradiated. In accordance with the method of the invention, the article to be irradiated is not exposed to ultra-violet radiation until the ultra-violet radiation sources of the apparatus have reached a maximum level of radiation output. Additionally, the apparatus is constructed and arranged so that the ultra-violet radiation impinging on the article can be instantly stopped thereby permitting a precise determination of the radiation dose to which the article has been exposed. To accomplish this, a source of radiation is disposed proximate the support that supports the article and a novel shutter arrangement is disposed between the support and the source of radiation for positively preventing any radiation from reaching the article until the shutter apparatus is moved to an open position.

### BACKGROUND OF THE INVENTION

1. Field of the Invention

The present invention relates generally to apparatus for irradiating various types of articles. More particularly, the invention concerns an apparatus for irradiating solid and fluid materials with ultra-violet radiation from a radiation source such as a plurality of ultra-violet lamps.

2. Discussion of the Prior Art

Ultra-violet radiation is widely used in industry and science for sterilization through inactivation of micro-organisms, for inducing and promoting various type of photo chemical reactions and for controllably exposing various type of photosensitive materials. By way of example, U.S. Pat. No. 3,936,186 issued to Boland et al discloses an apparatus for exposing diazo printing plates and the like of the character that are used in the graphic arts field. Similarly, U.S. Pat. No. 5,288,647 issued Zimlich, Jr. et al relates to a method by which polynucleotide specimens can be irradiated particularly for the purpose of fixing them to a substrate. The method of this latter mentioned patent uses an apparatus having a chamber that includes a repository for the specimen, an ultra-violet lamp fixture disposed within the chamber to illuminate the repository, and an energy detector that can provide an indication of the total ultra-violet dose received on the repository. The approach also includes a control circuit for de-energizing the lamp fixture upon detection of a selected or predetermined total ultra-violet dose received by the depository.

The Boland et al patent discloses an apparatus for exposing printing plates that includes a frame, a plurality of tubular lamps mounted within the frame, a bed disposed within the frame for supporting the printing plates, and a plurality of radiation sensors mounted adjacent the lamp. The apparatus also includes a plurality of control circuits that comprise means for energizing the lamps in response to a start signal and de-energizing the lamps when the lamp has provided a predetermined quantity of radiation as measured by irradiation sensors.

The prior art apparatus for irradiating articles such as those described in the Boland et al and Zimlich, Jr. et al patents control the amount of radiation to which the article is exposed by energizing the radiation source through appropriate control circuitry and then de-energizing the radiation source after a sensor has determined that the desired ultra-violet dose has been received by the specimen.

As will be better understood from the discussion that follows, the apparatus of the present invention controls the ultra-violet dose received by the article that is contained within the device housing in a different and highly novel manner. More particularly, in the apparatus of the invention hereinafter described, the ultra-violet dose is controlled by a novel shutter means that is disposed intermediate the source of radiation and the articles to be irradiated. This unique means of control enables greater control of the total dose exposure since the specimen is not exposed to the ultra-violet radiation until the lamps have reached their maximum radiation output. Similarly, after the desired radiation dose has been achieved, the shutter means instantaneously blocks the radiation. Stated another way, by knowing the amount of radiation emitted by the radiation sources and by precisely controlling the opening and closing of the shutter means, the dose of irradiation received by the article within the dosing chamber can be precisely controlled as a function of time. This greatly simplifies both the method and apparatus for irradiating the article that is to be irradiated.

### SUMMARY OF THE INVENTION

It is an object of the present invention to provide a method and apparatus for precisely controlling the area and accuracy of total radiation dose exposure of an article to be irradiated.

Another object of the invention is to provide a method and apparatus of the aforementioned character in which the article to be irradiated is not exposed to ultra-violet radiation until the ultra-violet radiation sources of the apparatus have reached a maximum level of radiation output.

Another object of the invention is to provide a method and apparatus as described in the preceding paragraph in which following the desired ultra-violet dosing of the article, the ultra-violet radiation impinging on the article can be instantly stopped thereby permitting a precise determination of the radiation dose to which the article has been exposed.

Another object of the invention is to provide an apparatus for irradiating an article that includes a support for supporting the article to be irradiated, a source of radiation disposed proximate the support and a novel shutter arrangement disposed intermediate the support and the source of radiation for positively preventing any radiation from reaching the article until the shutter apparatus is moved to an open position.

Another object of the invention is to provide an apparatus of the character described in the preceding paragraph in which the shutter mechanism also functions to block further irradiation of the article by substantially instantaneously closing the shutter mechanism.

Another object of the invention is to provide an apparatus as described in the preceding paragraph which includes control means operably associated with the shutter mechanism for opening and closing the shutter mechanism either at prescribed times or upon a determination having been made through the use of sensor means that the article being irradiated has received the desired dose of radiation.

Another object of the invention is to provide an apparatus of the class described in which various types of ultra-violet lamps can be installed, including shortwave ultra-violet lamps, longwave ultra-violet lamps, broad band ultra-violet lamps, blue/UV spectrum lamps and combinations thereof.

Another object of the invention is to provide an apparatus of the class described which is of simple construction, is highly reliable in operation and is easy to use by relative untrained operators.

### BRIEF DESCRIPTION OF THE DRAWINGS

FIG. 1 is a generally perspective view of one form of the apparatus of the invention partly broken away to show internal construction.

FIG. 2 is a generally perspective view of the radiation source and shutter control subassembly of the apparatus.

FIG. 3 is an enlarged, cross-sectional view taken along lines 3-3 of FIG. 2.

FIG. 4 is a cross-sectional view taken along lines 4-4 of FIG. 3.

FIG. 5 is a generally perspective view similar to FIG. 2 but showing the shutter component of the subsystem in an open configuration.

FIG. 6 is a cross-sectional view taken along lines 6-6 of FIG. 5.

FIG. 7 is a cross-sectional view taken along lines 7-7 of FIG. 6.

FIG. 8 is a generally perspective, fragmentary view of one form of the shutter operating system of the invention.

FIG. 9 is a side-elevational, fragmentary, cross-sectional view further illustrating the operating means for operating the shutter system of the apparatus of the invention.

FIGS. 10 and 11, when considered together, comprise a diagrammatic view illustrating the manner of operation of the apparatus of the invention.

FIG. 12 is a generally perspective view of a lamp array superimposed over an alternate form of shutter mechanism of the apparatus.

FIGS. 13A and 13B, when considered together, comprise a side-elevational view of the apparatus shown in FIG. 11.

FIG. 14 is a cross-sectional view taken along lines 14-14 of FIG. 13A.

FIGS. 15A and 15B, when considered together, comprise a side-elevational view similar to that shown in FIGS. 13A and 13B, but showing the shutter mechanism in a closed position.

FIG. 16 is a cross-sectional view taken along lines 16-16 of FIG. 15A

FIG. 17 is a generally perspective bottom view of the alternate form of shutter mechanism.

FIG. 18 is a generally perspective bottom view similar to FIG. 17, but showing the shutter mechanism in a closed position.

### DESCRIPTION OF THE INVENTION

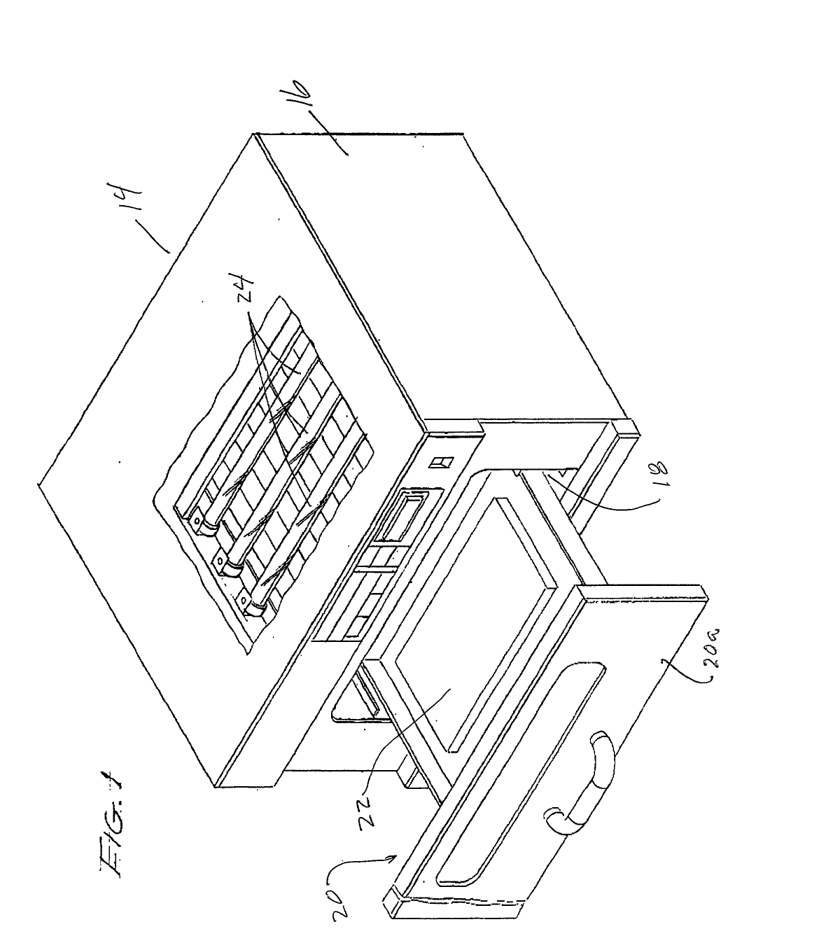

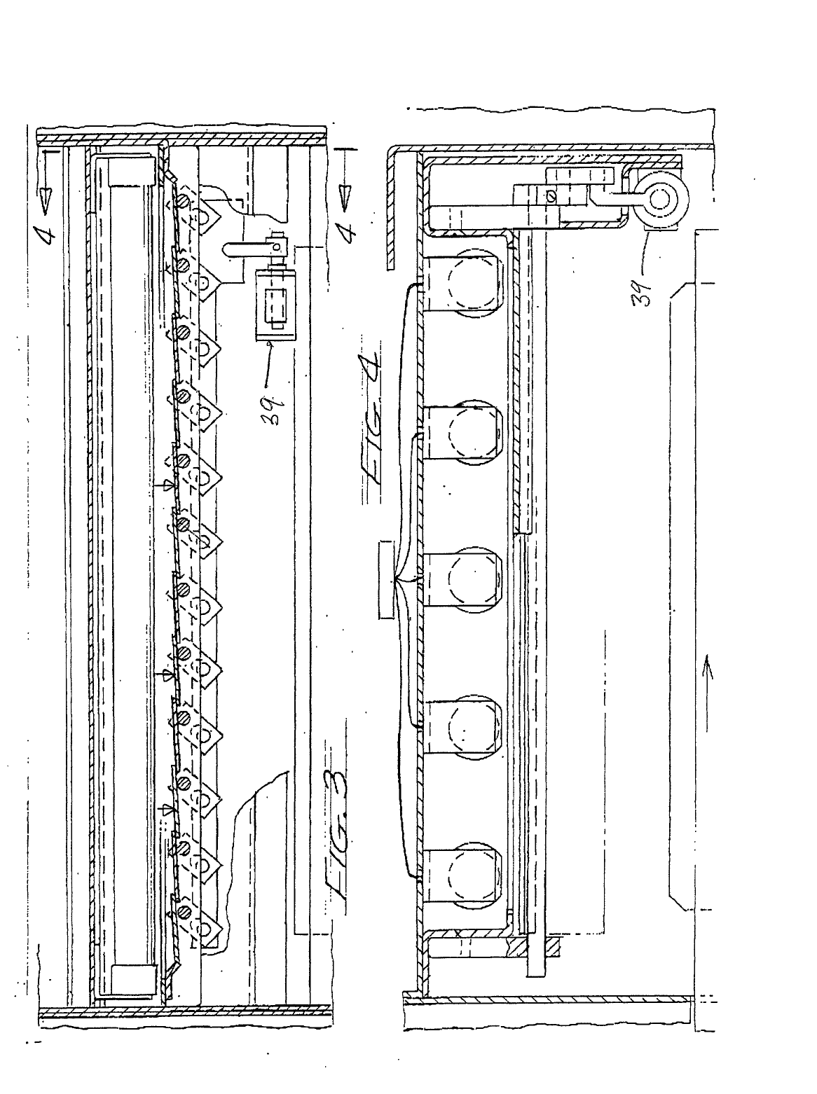

Referring to the drawings and particularly to FIGS. 1 through 5, one form of the irradiation apparatus of the present invention is there illustrated and generally designated by the numeral 14. The apparatus of this form of the invention comprises the housing 16 having an internal chamber 18 that is accessible by a pullout drawer assembly 20. Drawer assembly 20 includes a support platform 22 that can be disposed within chamber 18 of housing 16 when the drawer assembly is in its inward position. Support 22 is adapted to carry a specimen that is to be irradiated as, for example, a ploynucleocide.

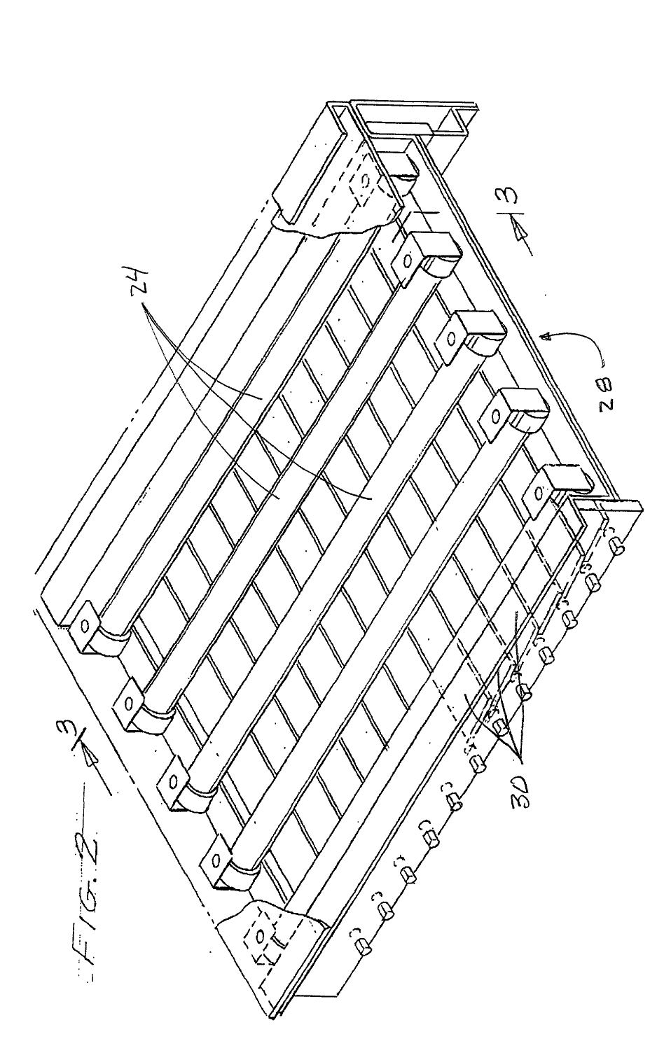

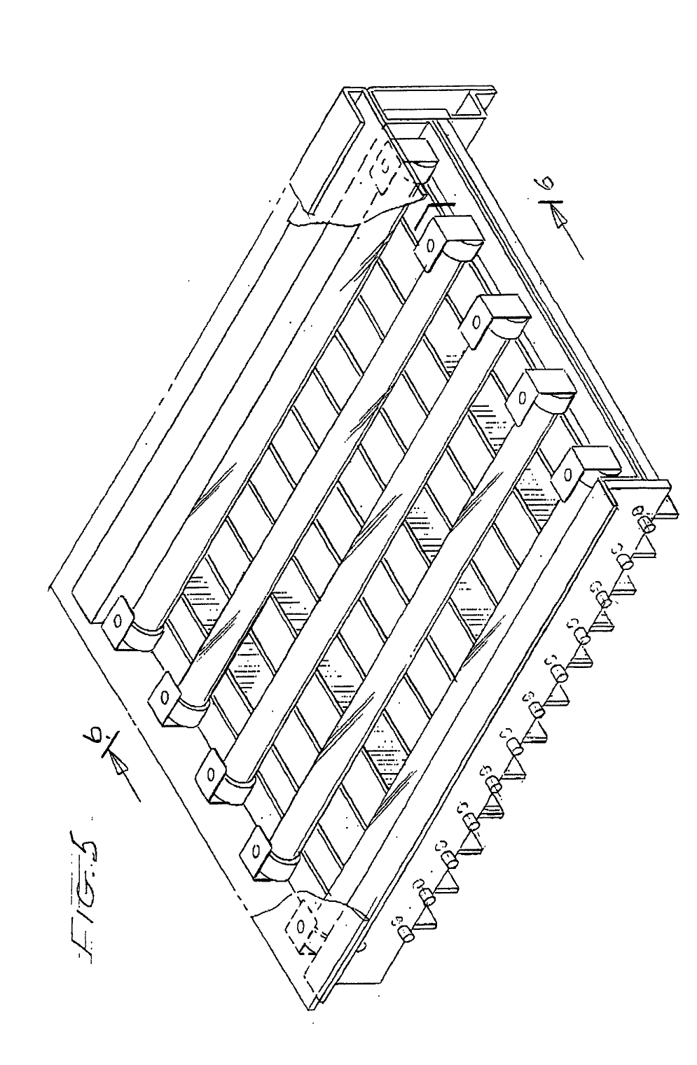

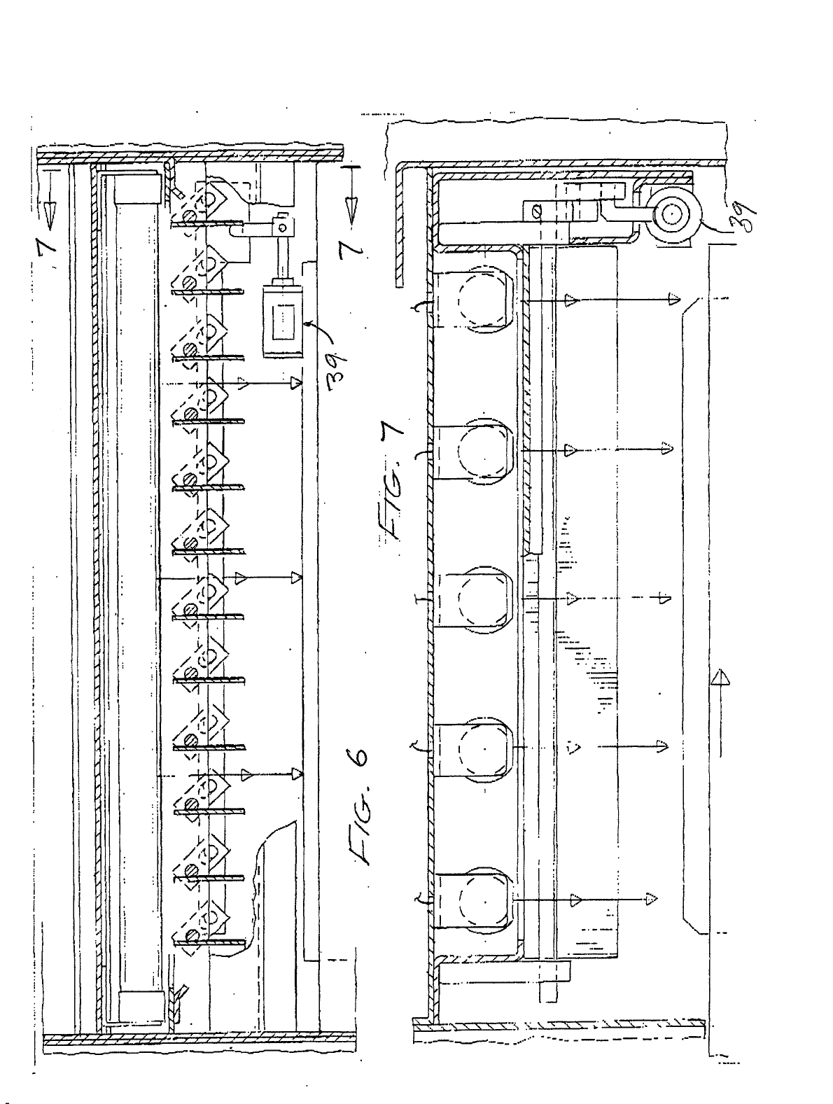

Also disposed within the upper portion of housing 16 is a source of irradiation that is spaced apart from source 22. As best seen in FIG. 2, the source of irradiation here comprises a course of ultraviolet radiation provided in the form of a plurality of spaced-apart, ultraviolet-light-emitting lamps 24. Positioned between the array of lamps 24 and support 22 is the highly novel shutter means of the invention which is adapted for movement between a first closed position blocking irradiation of the article carried on the support platform 22 and a second open position permitting irradiation of the article. More particularly, when the shutter means is in the closed position illustrated in FIG. 3, irradiation from the irradiation source is blocked from reaching support platform 22. Conversely, when the shutter means is in the open position shown in FIG. 6 radiation from the planar array of ultraviolet-emitting lamps can uniformly reach the support platform and the specimen emplaced thereon.

Referring particularly to FIG. 2, the shutter means of the present form of the invention comprises a supporting frame 28 that is mounted within housing 16 proximate the planar array of lamps 24. Pivotally connected to supporting frame 28 is a plurality of vanes 30. Vanes 30 are movable between a first shutter-closed position as shown in FIG. 3 to a second shutter-open position shown in FIG. 6.

The shutter operating means, which here comprises a solenoid assembly 32 (FIG. 3), is operably associated with control means for controlling the opening and shutting of the vanes of the shutter means. The control means comprises a timer which can be set for a particular time interval between energization of the array of ultraviolet-emitting lamps and the opening of the shutter means.

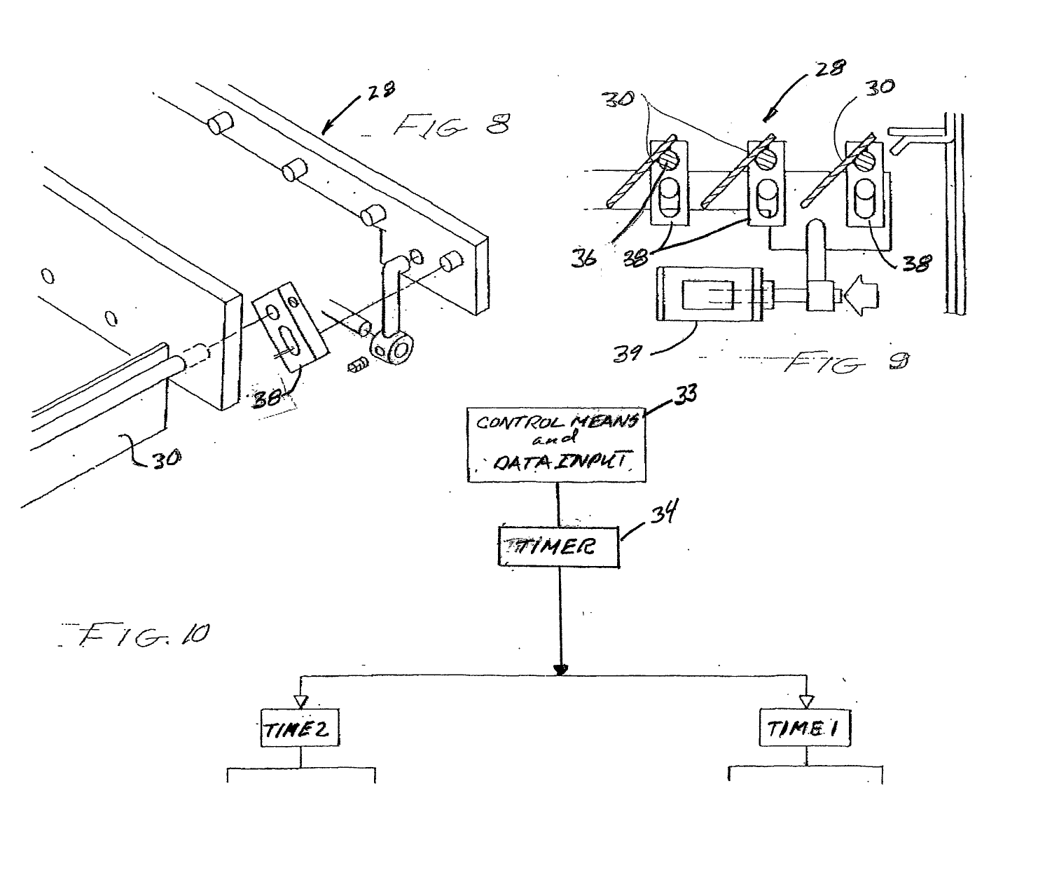

As best seen in FIGS. 8 and 9, each of the vanes means 30 is affixed to an operating rod 36 that is mounted for rotation within the frame assembly 28. Affixed to each of the elongated operating rods 32 is a block-like operating members 34. As indicated in FIGS. 3 and 6, the block-like operating members 38 are movable from a first position shown in FIG. 3, wherein the vanes are in a closed position, to a second position shown in FIG. 6 wherein the vanes are in an open position. It is to be understood that various mechanisms of a character well known to those skilled in the art could be used to pivot vanes 30 from an open position to a closed position. However, the use of the solenoid construction 39 illustrated in the drawings has proven satisfactory for the purpose.

As indicated in FIG. 10, the control means of the invention includes data input means that are operably associated with the timer 34 and can be used to set the first and second control times, the character of which will presently be described.

Positioned between the array of lamps 24 and support 22 is the highly novel shutter means of the invention which is adapted for movement between a first closed position blocking irradiation of the article carried on the support platform 22 and a second open position permitting controlled irradiation of the article. More particularly, when the shutter means is in the closed position illustrated in FIG. 3, radiation from the irradiation source is blocked from reaching support platform 22. Conversely, when the shutter means is in the open position shown in FIG. 6 radiation from the planar array of ultraviolet-emitting lamps can uniformly reach the support platform and the specimen emplaced thereon.

Referring particularly to FIG. 2, the shutter means of one form of the invention comprises a supporting frame 28 that is mounted within housing 16 proximate the planar array of lamps 24. Pivotally connected to supporting frame 28 is a plurality of blocking elements or vanes 30. Vanes 30 are pivotally movable between a first shutter-closed position as shown in FIG. 3 to a second shutter-open position shown in FIG. 6.

The shutter operating means, which here comprises a solenoid assembly 32 (FIG. 3), is operably associated with timer means 34 and control means 33 for controlling the opening and shutting of the blocking elements or vanes 30 of the shutter means. As depicted in FIG. 10, the control means is operably associated with the timer 34 which, in a manner presently to be described, can be set for a particular first time interval between energization of the array of ultraviolet-emitting lamps and the opening of the shutter means.

As best seen in FIGS. 8 and 9, each of the vanes 30 is affixed to an operating rod 36 that is mounted for rotation within the frame assembly 28. Affixed to each of the elongated operating rods 32 is a block-like operating members 34. As indicated in FIGS. 3 and 6, block-like operating members 38 are movable from a first position shown in FIG. 3, wherein the vanes 30 are in a closed position, to a second position shown in FIG. 6 wherein the vanes are in an open position. It is to be understood that various mechanisms of a character well known to those skilled in the art could be used to pivot vanes 30 from an open position to a closed position. However, the use of the solenoid construction 39 illustrated in the drawings has proven satisfactory for this purpose.

During operation of the apparatus of the invention, the data input means is used to input to the timer means a signal corresponding to a first selected interval of time during which the shutter means is to remain closed following the energization of the array of ultraviolet lamps. This time delay is necessary to enable the lamps to reach their full energization level prior to opening the shutter means to expose the specimen disposed within the housing 16 to the radiation. The data input means is also used to establish a second interval of time during which the shutter means is to remain open, which, of course, corresponds to the interval of time during which the article being irradiated is exposed to the ultraviolet radiation. Following the completion of the second interval of time, the shutter means will automatically close thereby blocking any further radiation of the specimen. In this way, the amount of radiation received by the specimen can be accurately and precisely controlled.

The control means and the data input means can take several forms, as for example, a microprocessor or digital device that permits measurement and calculation of the UV intensity at the support; measurement and calculation of the intensity of the UV lamps; measurement and calculation of the intended time of exposure assuming that the intensity of the lamps is known; and permits setting of the timer for operation of the shutter means at selected time periods.

Since the shutters do not open until the planar array of ultraviolet lamps reaches the maximum radiation level and since the irradiation time is positively controlled by the opening and closing of the shutter means, the specimen to be irradiated can be irradiated with a precise dose of ultraviolet radiation. This important feature is not found in the apparatus of the prior art wherein radiation is typically controlled by sensors located proximate the specimen being irradiated.

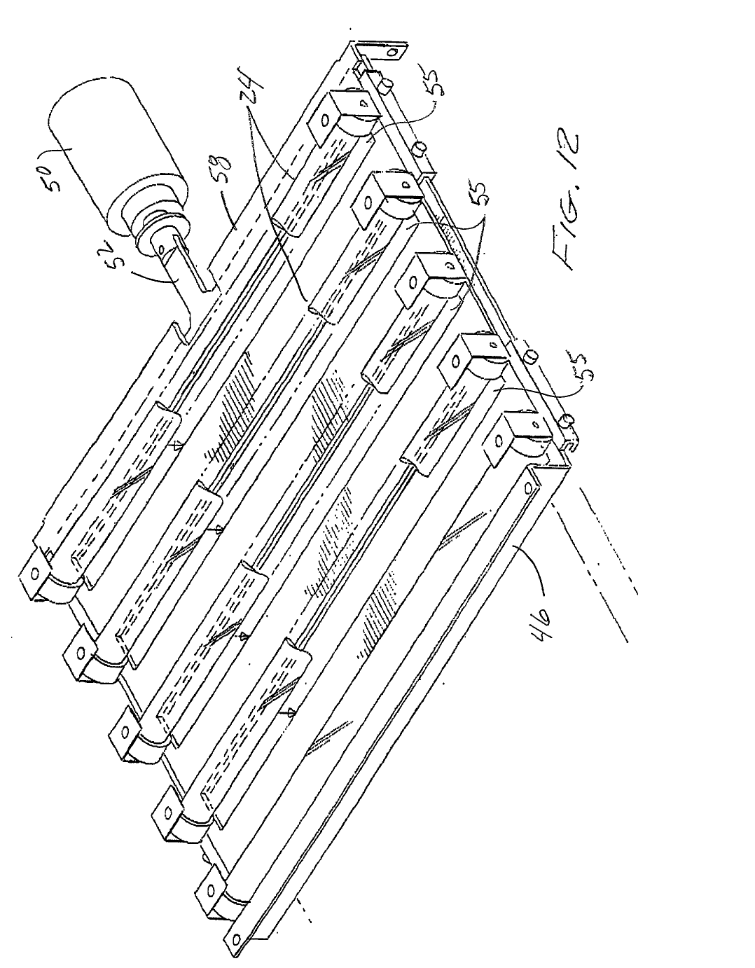

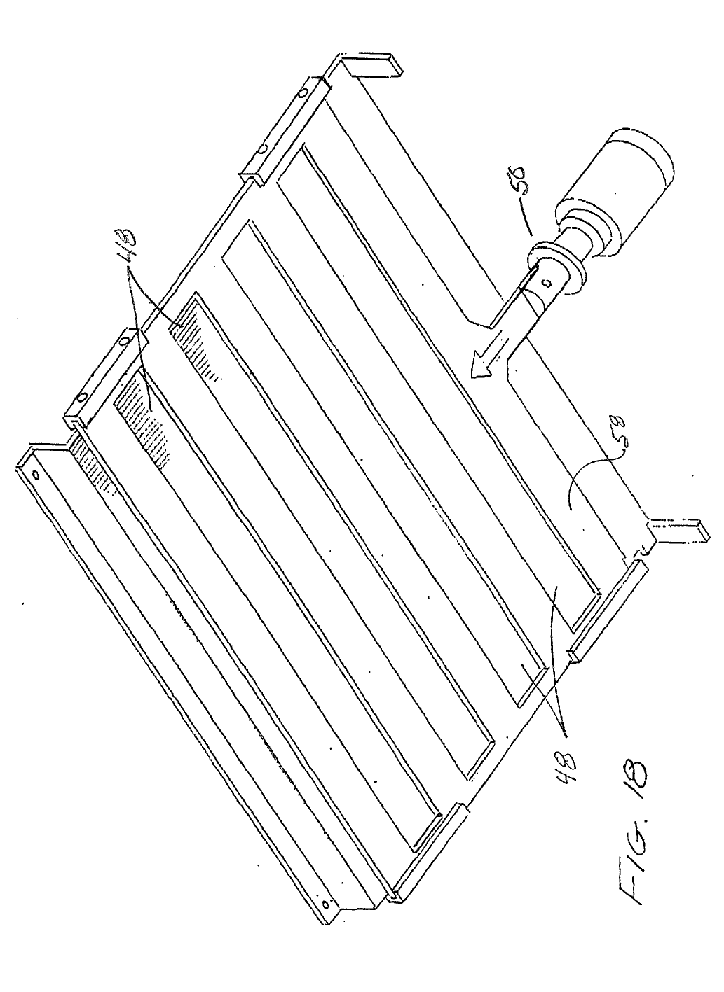

Turning now to FIGS. 12 through 18, an alternate form of shutter means of the invention is there shown. This alternate form of shutter means is also adapted for movement between a first closed position blocking irradiation of the article carried on the support platform 22 and a second open position permitting controlled irradiation of the article. Referring particularly to FIG. 12, this alternate form of shutter means comprises a supporting frame 46 that is mounted within housing 16 proximate the planar array of lamps 24. Slidably connected to supporting frame 46 is a panel assembly 48 that includes a plurality of blocking elements or panels 48a (FIG. 18). Panel assembly 48 is slidably movable between the first shutter-closed position as shown in FIG. 18 to a second shutter-open position shown in FIG. 17.

The shutter operating means of this alternate form of shutter means comprises a solenoid assembly 50 that is connected to panel assembly 48 and is also operably associated with timer means 34 and control means 33 for controlling the opening and closing of the blocking elements or panels 48a of the shutter means.

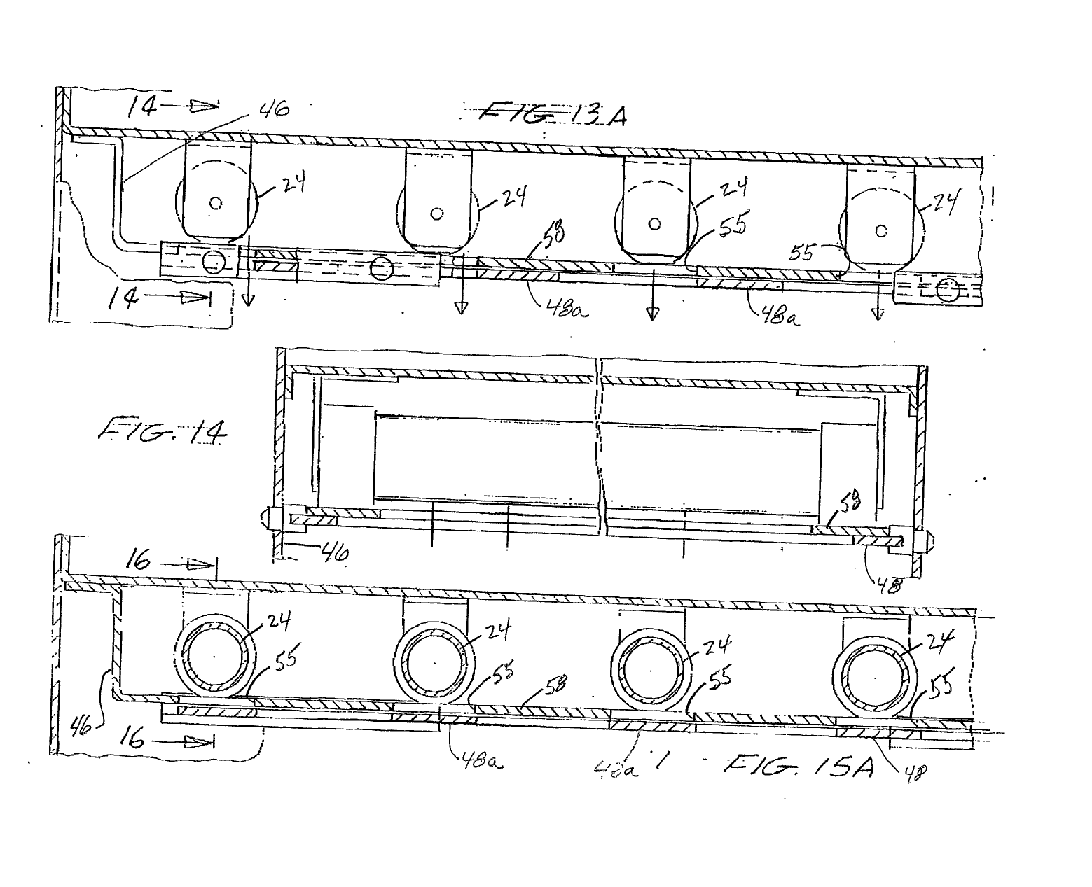

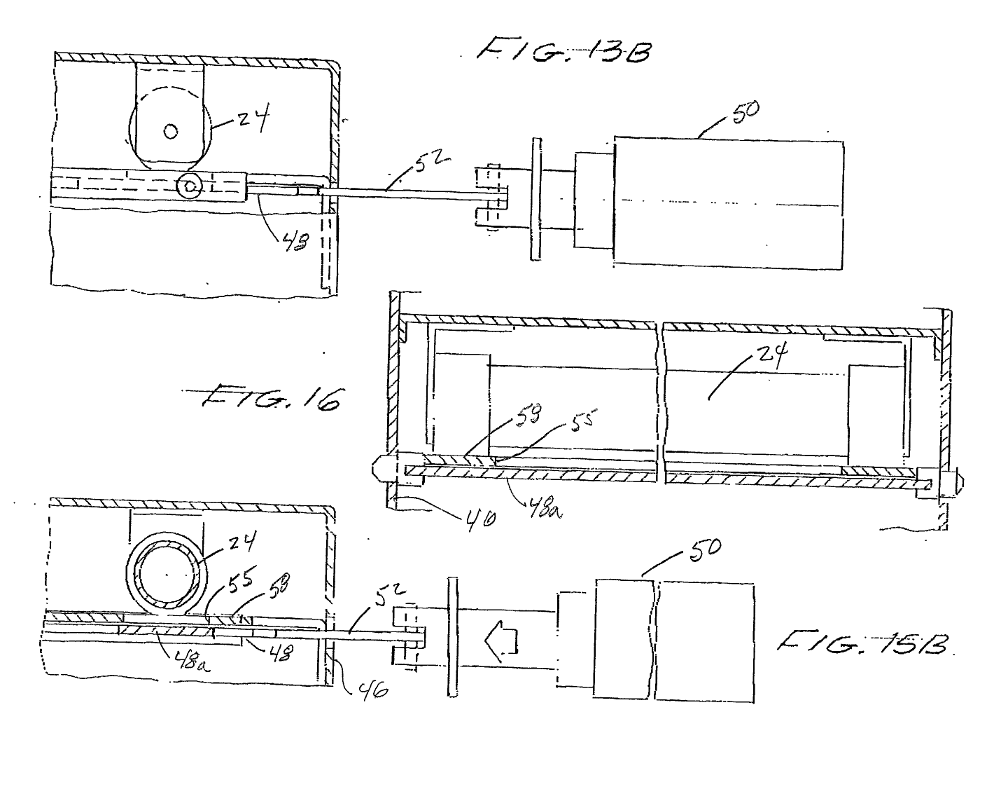

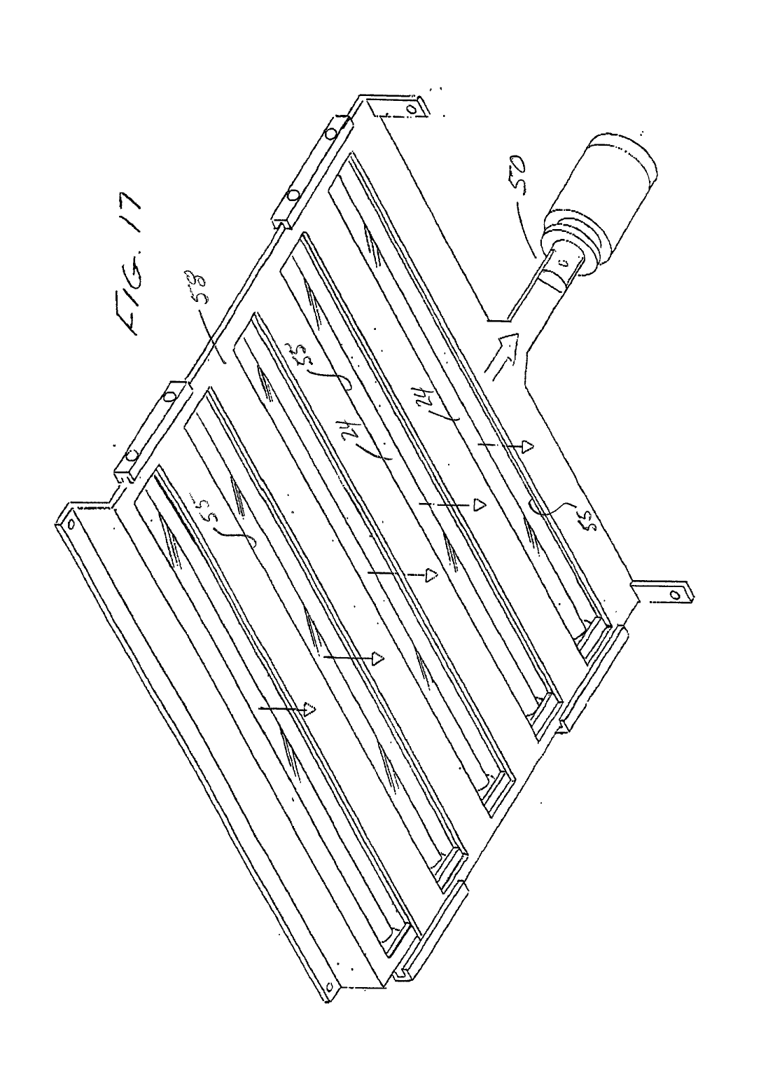

As best seen in FIGS. 13A, 13B, 15A and 15B, panel assembly 48 is affixed to an operating rod 52 that is mounted for reciprocal movement within frame assembly 46. More particularly, when the solenoid assembly 50 moves the operating rod 52 from the outward position shown in FIG. 13B to the inward position shown in FIG. 15B, panels 48a will be moved beneath a series of openings 55 formed in a top plate 58 that forms a part of frame assembly 46. It is apparent that with the panels in the position shown in FIG. 15B, the specimen disposed on support 22 will be totally shielded from radiation from lamps 24. As was the case in the earlier described embodiment, various mechanisms of a character well known to those skilled in the art could be used to move panel assembly 48 from an open position to a closed position. However, the use of the solenoid construction 52 illustrated in the drawings has proven satisfactory for this purpose.

During operation of this latest embodiment of the apparatus of the invention, the data input means is used to input to the timer means a signal corresponding to a first selected interval of time during which the shutter means is to remain closed following the energization of the array of ultraviolet lamps. The data input means is also used to establish a second interval of time during which the shutter means is to remain open, which, of course, corresponds to the interval of time during which the article being irradiated is exposed to the ultraviolet radiation. Following the completion of the second interval of time, the shutter means will automatically close thereby blocking any further radiation of the specimen. In this way, the amount of radiation received by the specimen can be accurately and precisely controlled.

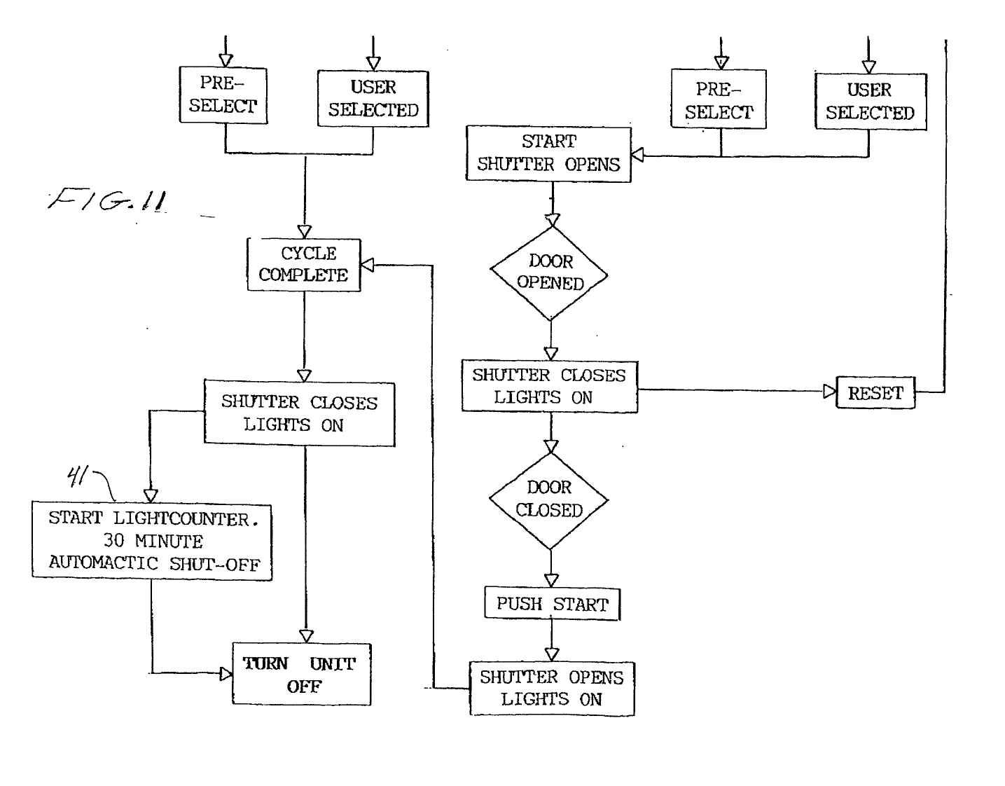

With the shutter means of the invention in the closed position, The first step in the accomplishment of the method of the invention involves determining a first time corresponding to the time required from initial energization of the source of radiation to achieving maximum radiation output from the source of radiation. Using the data input means, this first time is then communicated to the timer. As indicated in FIG. 10, the first time can be preselected or it can be selected by the user in real time. Next, the specimen to be irradiated is placed on the support 22. This is accomplished by sliding the drawer assembly from a first inward position to the second outward position shown in FIG. 1. After the specimen has been placed on support 22, the drawer assembly is once again moved into its first inward position. With the specimen appropriately positioned on support 22. a second time period is calculated. This second time period corresponds to the time period required to provide the desired dose of radiation to the specimen to be irradiated and, of course, depends on the nature of the specimen and the purpose for its irradiation. Once again, this second time can be communicated to the timer as a preselected time or alternatively it can be selected in real time by the user. Following the determination of the first and second time periods, the control means is used to appropriately energize the source of radiation. At the expiration of the first time period, and with the source of radiation at its maximum radiation level, the timer means is used to energize the solenoid assembly 39 in the manner to move the shutter means to the second open position as indicated in the right end portion of FIG. 11. The shutter means will remain in this second open position until the expiration of said second time period at which time the cycle is completed and timer is used to once again energize the solenoid assembly in a manner to move the shutter means to the first, closed position blocking further radiation of the specimen. As indicated in the right end portion of FIG. 11, as a safety measure, should the door be accidentally opened during the radiation cycle, the timer will automatically close the shutter. Once the door is again closed, the timer can be reset to start the cycle again. Upon restarting the cycle through use of the push start, the shutter will remain closed until the expiration of the first time period at which time the shutter will once again open to start the radiation cycle that will conclude at the end of the second time period. As indicated in the lower left hand portion of FIG. 11, after the cycle is completed and the shutter is closed, a light counter 41 will be activated. After a suitable time period, such as 30 minutes, the light counter 41 will automatically shut off the lights and de-energize the unit.

Having now described the invention in detail in accordance with the requirements of the patent statutes, those skilled in this art will have no difficulty in making changes and modifications in the individual parts or their relative assembly in order to meet specific requirements or conditions. Such changes and modifications may be made without departing from the scope and spirit of the invention, as set forth in the following claims.

I claim:

1. An apparatus for irradating an article comprising:
(a) a housing;
(b) a support disposed within said housing for supporting the article;
(c) a source of radiation disposed within said housing at a spaced apart location from said support;
(d) shutter means disposed intermediate said support and said source of radiation for movement between a first, closed position blocking irradiation of the article and a second open position permitting irradiation of the article; and
(e) control means for controlling said shutter means.

2. The apparatus as defined in claim 1 in which said source of radiation comprises a source of ultraviolet radiation.

3. The apparatus as defined in claim 1 in which said shutter means comprises:
(a) a supporting frame mounted within said housing proximate said planar array; and
(b) a plurality of blocking elements connected to said supporting frame for movement between a first, shutter closed position to a second, shutter open position.

4. The apparatus as defined in claim 1 further including a timer operably associated with said control means and with said shutter means for moving said shutter means between said first and second positions at selected intervals of time.

5. The apparatus as defined in claim 4 in which said control means comprises data input means operably associated with said timer for setting said timer.

6. An apparatus for irradiating an article comprising:
(a) a housing;
(b) a support disposed within said housing for supporting the article;
(c) a source of radiation disposed within said housing at a spaced apart location from said support, said source of radiation comprising a plurality of ultraviolet emitting lamps mounted within said housing;
(d) shutter means disposed intermediate said support and said source of radiation for movement between a first, closed position blocking irradiation of the article and a second open position permitting irradiation of the article, said shutter means comprising:
(i) a supporting frame mounted within said housing proximate said planar array; and
(ii) a plurality of blocking elements connected to said supporting frame for movement between a first, shutter closed position to a second, shutter open position; and
(e) control means operably associated with said shutter means for controlling said shutter means.

7. The apparatus as defined in claim 6 further including a timer operably associated with said shutter means and with said control means.

8. The apparatus as defined in claim 7 in which said shutter means further comprises shutter operating means operably associated with said timer for moving said vanes from said first shutter open position to said second shutter closed position upon the passage of a selected interval of time.

9. The apparatus as defined in claim 8 in which said shutter operating means comprises a solenoid.

10. The apparatus as defined in claim 8 in which said control means comprises data input means operably associated with said timer for controlling the operation of said timer.

11. The apparatus as defined in claim 8 in which said blocking elements comprise a plurality of vanes pivotally connected to the said supporting frame for movement between a first shutter open position and a said second shutter closed position.

12. The apparatus as defined in claim 8 in which said blocking elements comprise a plurality of panels connected to the said supporting frame for movement between a first shutter open position and a said second shutter closed position.

13. A method for irradating an article using an apparatus comprising a housing, a support disposed within the housing for supporting the article, a source of radiation disposed within the housing at a spaced apart location from the support, shutter means disposed intermediate the support and the source of radiation for movement between a first, closed position blocking irradiation of the article and a second open position permitting irradiation of the article, and control means for energizing the source of radiation and for controlling the shutter means, the method comprising the steps of:
(a) placing the article to be irradiated on the support;
(b) determining the time period of time that the article is to be irradiated;
(c) using the control means energizing the source of radiation;
(d) using the control means, moving the shutter means to the second open position; and
(e) at the end of said time period, moving the shutter means to the first, closed position.

14. The method as defined in claim 13 including the further steps of determining the time period required from initial energization of the source of radiation to achieving maximum radiation output from the source of radiation and, after energizing the source of radiation, maintaining the shutter means in the first closed position until the expiration of said time period required from initial energization of the source of radiation to achieving maximum radiation output from the source of radiation.

15. A method for irradating an article using an apparatus comprising a housing, a support disposed within the housing for supporting the article at a spaced apart location from the support, shutter means disposed intermediate the support and the source of radiation for movement between a first, closed position blocking irradiation of the article and a second open position permitting irradiation of the article, a timer operably associated with the shutter means and a control means for energizing the source of radiation and for controlling the timer, the method comprising the steps of:
(a) placing the specimen to be irradiated on the support;
(b) determining the elapsed time from energizing the source of radiation to emission of maximum radiation by the source of radiation to define a shutter open time;
(c) using the control means, energizing the source of radiation;
(d) at the shutter open time, using the timer means to move the shutter means to the second open position;
(e) determining the length of time during which the article is to be irradiated to define a shutter close time; and
(f) at the shutter close time, using the timer means to move the shutter means to the first closed position.

16. The method as defined in claim 15 in which the support is mounted on a drawer having a door and in which the method includes the further step of closing said shutter means upon opening the door.

17. The method as defined in claim 15, including the further step of, following movement of the shutter means to the first closed position, deenergizing the source of radiation.
